(19) United States
(12) Patent Application Publication
(10) Pub. No.: US 2006/0276460 A1
(43) Pub. Date: Dec. 7, 2006

(54) Use of piperazine phenothiazine derivatives in the manufacture of a medicament with neuroprotector and/or neurotrophic effects on cns and/or pns

(76) Inventors: Noelle Callizot, Strasbourg (FR); Aline Appert Collin, Strasbourg (FR)

Correspondence Address: JENKINS, WILSON, TAYLOR & HUNT, P. A., 3100 TOWER BLVD, SUITE 1200, DURHAM, NC, 27707, US

(21) Appl. No.: 10/551,395

(22) PCT Filed: Apr. 23, 2004
(86) PCT No.: PCT/EP04/05183

§ 371 Date: Sep. 29, 2005

(30) Foreign Application Priority Data
Apr. 25, 2003 (EP) 03291024.2

(51) Int. Cl. A61K 31/5415

(52) U.S. Cl. 514/225.8

(57) ABSTRACT

This invention relates to a new use of piperazinephenothiazine derivatives and their pharmaceutically acceptable salts or esters in the manufacture of a medicament with neuroprotector and/or neurotrophic effects on CNS and/or PNS. The piperazine phenothiazine derivatives are selected from compounds of formula (I) wherein A represents a straight or branched alkylene chain of from 2 to 6 carbon atoms separating the nitrogen atoms linked thereto by at least two carbon atoms; R1 represents hydrogen, halogen, lower alkyl, lower alkoxy, lower alkanoyl, lower alkyl-mercapto, trifluoromethylmercapto, lower alkyl-sulfonyl (preferably methylsulfonyl), perfluoroalkyl of 1 to 3 carbon atoms; R2, R3, R4 and R5 each represent methyl, ethyl or hydrogen, R6 represents hydrogen, lower alkyl, hydroxy-lower-alkyl or aliphatic acyloxy-lower-alkyl having 1 to 4 carbon atoms in the acyloxy portion and I to 6 carbon atoms in the alkyl portion, CH₂—CH₂—O—R7 where R7 represents hydrogen, COR8 where R8 is a branched or straight chain alkyl radical of from seven to fourteen carbon atoms.

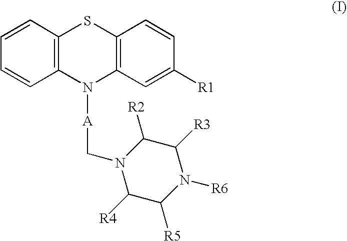

### FIELD OF THE INVENTION

This invention relates to a new use of piperazine phenothiazine derivatives and their pharmaceutically acceptable salts or esters in the manufacture of a medicament with neuroprotector and/or neurotrophic effects on CNS and/or PNS.

### BACKGROUND OF THE INVENTION

These compounds are known as major tranquilizers and neuroleptic drugs for their effects as relieving schizophrenic agitation, and maniacal behaviour. More particularly the piperazine series which includes trifluoroperazine, prochlorperazine, flufenazine are the most potent phenothiazine antipsychotic compounds. Flufenazine is marketed under the tradename Moditen® for its neuroleptic therapeutical effects. Fluphenazine and its hydrochloride salt or enanthate or decanoate ester form exerts activity at various levels of the CNS as well as on peripheral organ systems, which accounts for its antipsychotic action and side effects. Indirect evidence indicates that the antipsychotic effects of phenothiazines are linked to their effect in blocking dopamine and other catecholamine receptor sites.

### SUMMARY OF THE INVENTION

Surprisingly, the applicant has found that piperazine phenothiazine derivatives and more particularly flufenazine, are able to exert significant neuroprotective and neurotrophic effects. These new effects which could not be derived from actual flufenazine antipsychotic action have been highlighted during specific in vitro and in vivo model studies of CNS and PNS neuronal degeneration.

The invention relates to

1. Use of piperazine phenothiazine derivatives, or a pharmaceutically acceptable salt or ester thereof, in the manufacture of a medicament with neuroprotector and/or neurotrophic effects on CNS and/or PNS.

2. Use according to item 1 wherein the piperazine phenothiazine derivatives are selected from compounds of formula I 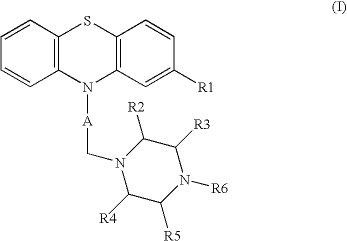
wherein

A represents a straight or branched alkylene chain of from 2 to 6 carbon atoms separating the nitrogen atoms linked thereto by at least two carbon atoms ;

R1 represents hydrogen, halogen, lower alkyl, lower alkoxy, lower alkanoyl, lower alkyl-mercapto, trifluoromethylmercapto, lower alkyl-sulfonyl (preferably methylsulfonyl), perfluoroalkyl of 1 to 3 carbon atoms;

R2, R3, R4 and R5 each represent methyl, ethyl or hydrogen, R6 represents hydrogen, lower alkyl, hydroxy-lower-alkyl or aliphatic acyloxy-lower-alkyl having 1 to 4 carbon atoms in the acyloxy portion and 1 to 6 carbon atoms in the alkyl portion, CH₂—CH₂—O—R7 where R7 represents hydrogen, COR8 where R8 is a branched or straight chain alkyl radical of from seven to fourteen carbon atoms;

in the manufacture of a medicament with neuroprotector and/or neurotrophic effects on CNS and/or PNS.

3. Use according to item 2 wherein

A represents ethylene, propylene or 2-methylpropylene;

R1 represents hydrogen, chloro, COCH₃, —CF₃;

R2, R3, R4 and R5 each represent hydrogen;

R6 represents CH₃, CH₂—CH₂—O—R7 where R7 represents hydrogen, COR8 where R8 is a straight chain alkyl radical of from seven to fourteen carbon atoms.

4. Use according to items 1 to 3, wherein the piperazine phenothiazine derivatives is flufenazine, or a pharmaceutically acceptable salt or ester thereof, in the manufacture of a medicament with neuroprotector and/or neurotrophic effects on CNS and/or PNS.

5. Use according to items 1 to 4, in the manufacture of a medicament for the treatment of central and/or peripheral neurodegenerative diseases.

6. Use according to items 1 to 5, in the manufacture of a medicament for the treatment of Parkinson's disease.

7. Use according to items 1 to 5, in the manufacture of a medicament for the treatment of Alzheimer's disease.

8. Use according to items 1 to 5, in the manufacture of a medicament for the treatment of peripheral neuropathy diseases.

9. Use according to item 8, in the manufacture of a medicament for the treatment of amyotrophic lateral sclerosis (ALS) diseases.

10. Use according to any one of items 1 to 9, wherein the medicament is for oral, rectal, subcutaneous, intramuscular or intravascular administration route.

11. Use according to any one of items 1 to 10, wherein the medicament comprises as active ingredient from 0.2 mg to 500 mg of the piperazine phenothiazine derivatives.

12. Use according to any one of items 1 to 11, wherein the medicament is administrated at doses comprised between 0.1 mg/kg to 10 mg/kg.

The invention provides also methods for the treatment of central and/or peripheral neurodegenerative diseases, of Parkinson's disease, of Alzheimer's disease, of peripheral neuropathy diseases or for the treatment of amyotrophic lateral sclerosis (ALS) diseases. The above methods comprise the administration to human or other animal subjects of an effective amount of piperazine phenothiazine derivatives compounds of formula I having neuroprotector and/or neurotrophic effects.

### BRIEF DESCRIPTION OF THE FIGURES

FIG. 1: flufenazine effects on spinal cord neurons survival

FIG. 2: protective effects on cortical neurons of flufenazine after glutamic acid intoxication and with maturation with BDNF.

FIG. 3: protective effects of flufenazine on mesencephalic neurons after MPP+ intoxication

FIG. 4: neurotrophic effects of flufenazine on cortical neurons

FIG. 5: neurotrophic effects of flufenazine on spinal cord neurons

FIG. 6: survival rate of SOD mice after oral administration of flufenazine.

### DETAILED DESCRIPTION

Thus, according to the present invention, there is provided the use of piperazine phenothiazine derivatives and a pharmaceutically acceptable salt and /or ester thereof, in the manufacture of a medicament with neuroprotector and neurotrophic effects on CNS and PNS

Piperazine phenothiazine derivatives are defined as compounds of the formula I

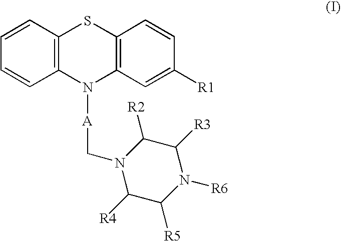

wherein

A represents a straight or branched alkylene chain of from 2 to 6 carbon atoms separating the nitrogen atoms linked thereto by at least two carbon atoms;

R1 represents hydrogen, halogen (preferably chloro), lower alkyl, lower alkoxy, lower alkanoyl (preferably COCH₃), lower alkyl-mercapto, trifluoromethylmercapto, lower alkyl-sulfonyl (preferably methylsulfonyl), perfluoroalkyl of 1 to 3 carbon atoms, preferably CF₃;

R2, R3, R4 and R5 each represent methyl, ethyl or hydrogen; R6 represents hydrogen, lower alkyl, hydroxy-lower-alkyl or aliphatic acyloxy-lower-alkyl having 1 to 4 carbon atoms in the acyloxy portion and 1 to 6 carbon atoms in the alkyl portion, CH₂—CH₂—O—R7 where R7 represents hydrogen, COR8 where R8 is a straight or branched chain alkyl radical of from seven to fourteen carbon atoms.

The terms “lower alkyl,” “lower alkoxy”, “lower alkanoyl” as employed herein include both straight and branched chain radicals of from 1 to 6 carbon atoms.

Preferably the piperazine phenothiazine derivatives used in the medicaments of the invention are selected from compounds of formula I wherein

A represents ethylene, propylene or 2-methylpropylene;

R1 represents hydrogen, chloro, COCH₃, CF₃;

R2, R3, R4 and R5 each represent hydrogen;

R6 represent CH₂—CH₂—O—R7 where R7 represents hydrogen, COR8 where R8 is a straight chain alkyl radical of from seven to fourteen carbon atoms.

More preferably, the piperazine phenothiazine derivatives used in the medicaments of the invention is 4-[3-[2-(trifluoromethyl)-10H-phenothiazin-10-yl]propyl]-1-pyperazine-ethanol (flufenazine) or salts and /or ester thereof.

These compounds and their chemical preparations under free bases form are disclosed in GB 829246, U.S. Pat. No. 3,058,979.

This invention also includes salts of the above defined bases formed with non-toxic organic and inorganic acids.

Such salts are easily prepared by methods known in the art and are disclosed in GB 829246, U.S. Pat. No. 3,058,979.

The invention also covers ester derivatives of the above compounds and their preparations are described in GB 833474 and U.S. Pat. No. 3,194,733.

Thus, these documents, GB 829246, U.S. Pat. No. 3,058,979, GB 833474 and U.S. Pat. No. 3,194,733 are hereby incorporated herein by references.

The medicaments of the invention are for treating central and peripheral neurodegenerative diseases as Parkinson's disease, Alzheimer's diseases or peripheral neuropathy diseases as particularly amyotrophic lateral sclerosis (ALS)diseases. The neuroprotective and neurotrophic effects of flufenazine were tested in vitro on primary neuronal cell cultures and in vivo, in animal model studies.

In vitro studies were first conducted on spinal cord motoneurons which are involved in peripheral neuropathy diseases as for example amyotrophic lateral sclerose (ALS) according to protocols described by Martinou J. C., Martinou I., Kato A. C. in Cholinergic differentiation factor (CDF/LIF) promotes survival of isolated rat embryonic motoneurons in vitro. Neuron 1992, 8(4) 737-744) and by Ometani A, Nomoto H, Nitta A, Furukawa Y, Furukawa S. in 4-Methylcatechol stimulates phosphorylation of Trk family neurotrophin receptors and MAP kinases in cultured rat cortical neuron. J Neurosci Res Nov. 1, 2002;70(3):335-9.

Further studies were conducted on cortical neurons intoxicated with glutamic acid according to the protocol described by Nilsen J. and Brinton R D in Impact of progestins on oestrogen-induced neuroprotection:synergy by progesterone and 19-norprogesterone and antagonism by medoxyprogesterone acetate. Endocrinology 143:205-212 2002.

Cortical neurons are involved in Alzheimer's disease and also in mesencephale neurons or dopaminergic neurons which are themselves involved in Parkinson's disease. (see also protocol described by Y. Mitsumotol, A. Watanabe, T. Miyauchi, F. Jimma, and T. Moriizumi in Stimulation of the regrowth of MPP+/damaged dopaminergic fibers by the treatment of mesencephalic cultures with basigin; J Neural Transm (2001) 108: 1127-1134).

In vivo studies were conducted on transgenic animals with ALS causing mutations, a model for neurodegenerative diseases according to protocols described by Gurney Me, Pu H, Chiu Ay, Dal Canto Mc, Polchow Cy, Alexander Dd, Caliendo J, Hentati A, Kwon Yw, Deng Hx, et al. in Motor neuron degeneration in mice that express a human Cu,Zn superoxide dismutase mutation. Science (1994) 264:1772-1775; and by Mohajeri Mh, Figlewicz Da, Bohn Mc in Selective loss of alpha motoneurons innervating the medial gastrocnemius muscle in a model of amyotrophic lateral sclerosis. Exp. Neurol. (1998) 150:329-336.

All these tests demonstrate that neuronal survival increase in the presence of several concentrations of flufenazine compared to the control without flufenazine and that flufenazine induces neuroprotective action after different intoxications.

The neurotrophic effect i.e. the neurite outgrowth with flufenazine was investigated on both cortical and spinal cord neuronal cultures according to the protocol described by Lucius R, Sievers J. in Postnatal retinal ganglion cells in vitro: protection against reactive oxygen species (ROS)-induced axonal degeneration by cocultured astrocytes Brain Res Dec. 16, 1996;743(1-2):56-62.

The results on neurite length as well as the percentage of cells with neurites quantified by careful microscopic inspection demonstrate the neurotrophic effect in the presence of several flufenazine concentrations compared to the control without flufenazine.

The in vivo test results demonstrate the improved animals survival with the administration of several doses of flufenazine compared to the control without flufenazine.

These medicaments can be administered by oral, rectal, subcutaneous, intramuscular or intravascular administration routes.

The medicaments according to the invention can be solids or liquids and be presented in the pharmaceutical forms commonly used in human medicine, such as for example, plain or sugar-coated tablets, gelatin capsules, granules, suppositories, injectable preparations, ointments, creams, gels; they are prepared according to the usual methods. The active ingredient(s) can be incorporated with the excipients usually used in these pharmaceutical compositions, such as talc, gum arabic, lactose, starch, magnesium stearate, cocoa butter, aqueous or non aqueous vehicles, fatty substances of animal or vegetable origin, paraffin derivatives, glycols, various wetting, dispersing or emulsifying agents, preservatives.

These compositions can in particular be presented in the form of a powder intended to be dissolved extemporaneously in an appropriate vehicle, for example apyrogenic sterile water.

The medicament can comprise as active ingredient from 0.2 mg to 500 mg of the piperazine phenothiazine derivatives.

The dose administered is variable according to the condition treated, the patient in question, the administration route and the product considered. It can be, for example, comprised between 0.01 mg and 50 mg per day by oral route in adults with flufenazine or also comprised between 0.1 mg and 10 mg per day by intramuscular or intravenous route.

### EXAMPLES

In vitro studies:

Cell Cultures Conditions:

Two types of primary neuronal cell cultures i.e. cortical and spinal cord neurons are isolated from 15-17 days old foetuses of female Wistar rats and cultured in neurobasal/B27 medium until cell differentiation.

Neuroprotection Essays;

All tests are conducted with their appropriate control.

### Example 1

Maintenance of neuronal survival and proliferation is a crucial process for the integrity of neurons. In the present study spinal cord motoneuron cell culture are used to assess whether or not flufenazine promote neuronal survival and proliferation.

Neuronal Survival and Proliferation

Cells cultures are incubated with different Fluphenazine N-Mustard dihydrochloride concentrations: 50, 100, 250 nmol/l.

Neuronal survival is then monitored at different time points; 2, 24, 48, 72, 96, 120 hours by counting the number of cells under microscope.

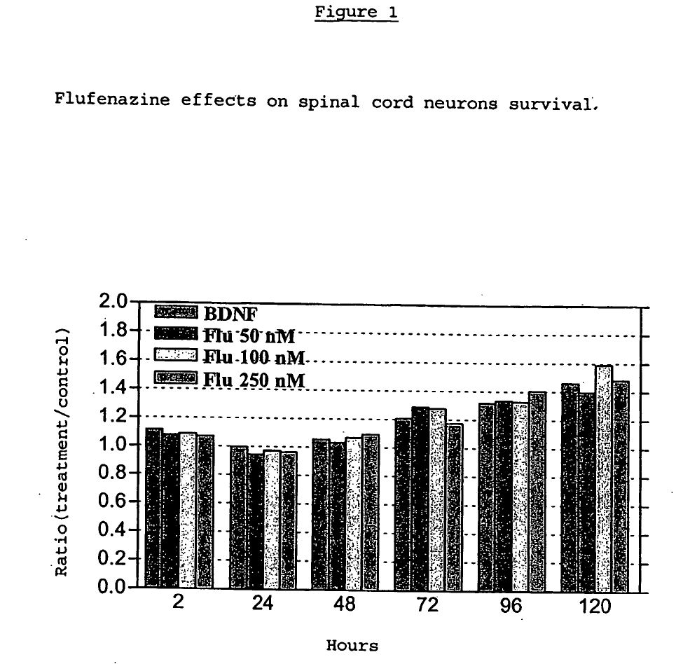

The results are depicted in FIG. 1:

the percentage of neuronal survival obtained when cells cultures are incubated with flufenazine is compared to cell cultures incubated alone only with 50 microgram's of brain derived neurotrophic factor (BDNF). The whole percentages increase regularly from 48 h until 120 h and the best survival results are obtained with flufenazine concentrations of 50 to 250 nmol/l.

### Example 2

Glutamic Acid Intoxication Assay:

The neuroprotective activity is assessed on glu-induced cortical neurons loss. The process of death is initiated by 10 min treatment of neuron cell cultures with neurotoxic concentration of glutamic acid at 100 micromol/l.

The ability of flufenazine to reverse the death process is achieved by subsequent exposure of cells to flufenazine concentrations of 100 and 200 nmol/l. The quantity of LDH released is used to estimate the degree of intoxication and the decrease of quantity released is proportional at cellular resistance to Glu neurotoxicity.

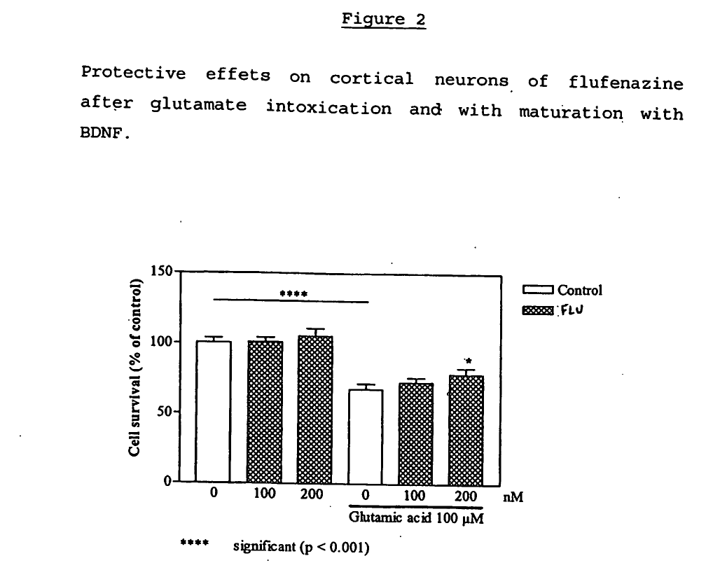

The results are depicted in FIG. 2 (right part):

the percentage of neuronal survival obtained when intoxicated cells cultures are incubated with flufenazine is compared to cell cultures without flufenazine.

With 200 nmol/l of flufenazine, a 10% increase is significantly observed.

In the left part of FIG. 2, it can be observed that flufenazine has no or very slight effect, on cell survival in the absence of neurotoxic compound.

### Example 3

MPP+ Intoxication Essay:

The neuroprotective activity is assessed on MPP+ induced mesencephalic neuron loss with flufenazine concentration of 250 nmol/l

The cells are intoxicated by 2 micromol of MPP+ as neurotoxic during 24 h. The cell culture is then treated with 250 nm/l of flufenazine. Reversed effects are observed after 48 hours.

These neuroprotective effects are measured by the increased number of TH positive cells or dopaminergic neurons (i.e. mesencephale neurons which contains, tyrosine hydroxylase (TH), a dopamine synthesis enzyme.

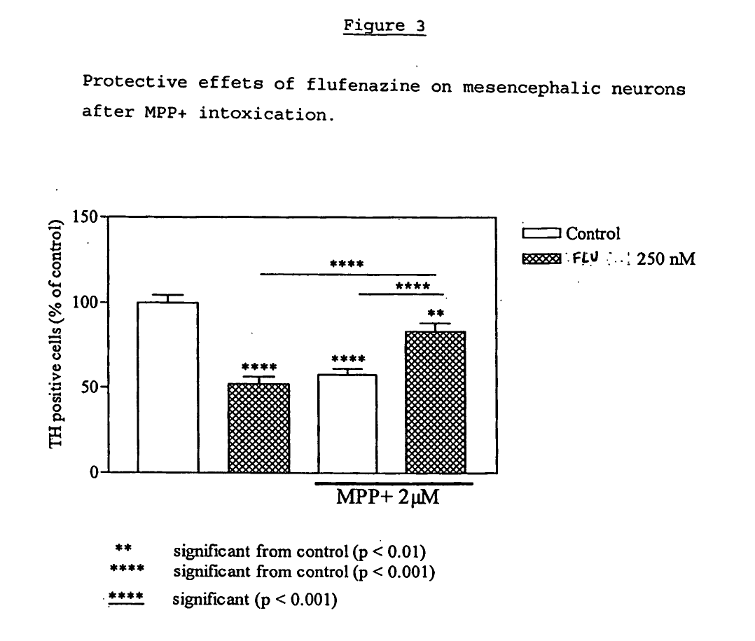

The results are depicted in FIG. 3 (right part):

The percentage of mesencephalic neuronal survival obtained when intoxicated cells cultures are incubated with flufenazine is compared to cell cultures without flufenazine.

With 250 nmol/l of flufenazine, a 30% increase of TH positives cells is significantly observed.

These results show that flufenazine (250 nmol/l) reverses MPP+ induced neuronal loss to the same extent as Riluzole (5 micromoles/l) a drug with established neuroprotective activity in this model (see reference Storch A, Burkhardt K, Ludolph A C, Schwarz J. Protective effects of riluzole on dopamine neurons: involvement of oxidative stress and cellular energy metabolism. J Neurochem December 2000;75(6):2259-69).

In the left part of FIG. 3, it can be confirmed that flufenazine according to its known extrapyramidal reactions has a negative effect (minus 50%) on the survival of this type of dopaminergic neurons.

In addition this assay also demonstrates longer neurite expansion than in control neurons at 250 nmol/l).

### Example 4

Neurotrophic Assays:

The ability of flufenazine to induce neurite outgrowth is investigated both in cortical and spinal cord neuronal cultures after 24 h exposure to flufenazine.

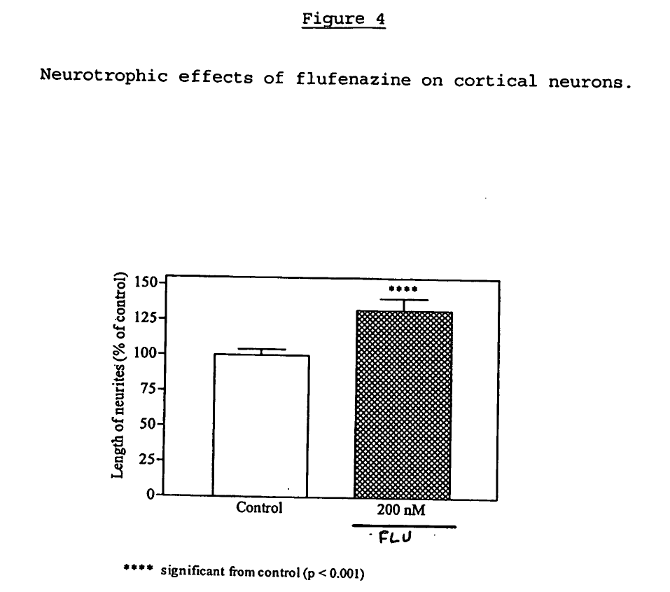

The neurite length as well as the percentage of cells with neurites are quantified by careful microscopic inspection. On cortex neurons type, the results are depicted in FIG. 4; the cortex neurotrophic effect of flufenazine (200 nmol/l) is expressed as an increase of 30% on neurites length compared to the neurites length in the control cortex neurons

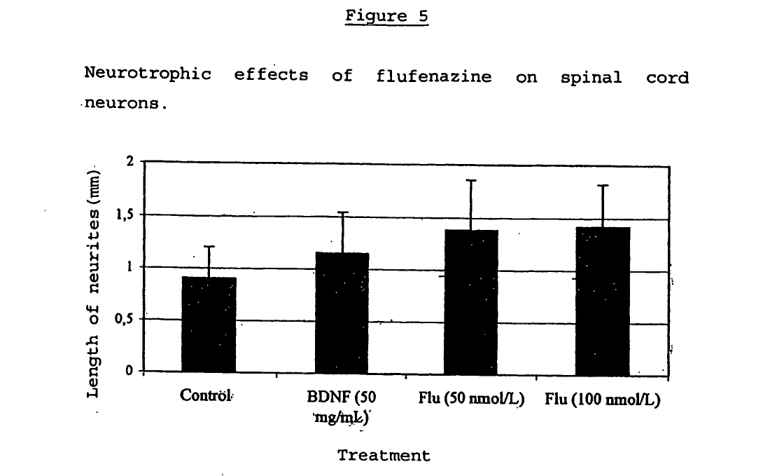

On spinal cord neurons, the results are depicted in FIG. 5:

The neurotrophic effect of flufenazine (50 nmol/l, 100 nmol/l) is expressed as an increase of 40 to 50% on neurites length compared to the neurites length in the control spinal cord neurons.

### Example 5

In vivo assays.

Animals and Flufenazine Treatment.

Mice are used at 4 months of age i.e. about 2 weeks before the appearance of the first symptoms.

They are genotyped for SOD1 gene using PCR method.

Animals are assigned into four groups to receive a daily oral administration of 1)saline as control, 2) 0.1 mg/kg, 3) 1 mg/kg, 4) 10 mg/kg of flufenazine, until death by weakness and paralysis occurs.

Survival rate was recorded on a daily basis.

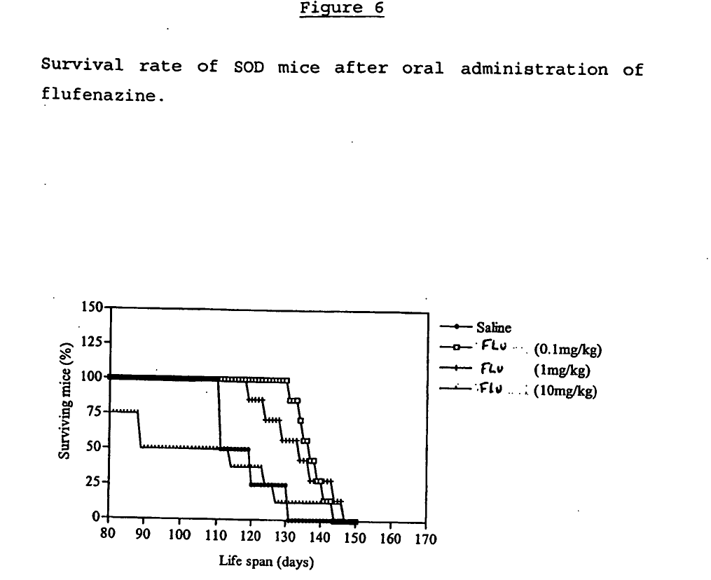

The results are depicted in FIG. 6:

Doses of 0.1 and 1 mg/kg flufenazine enhanced the survival of SOD mice as compared to that of the saline treated group. In contrast, higher dose (10 mg/kg) appeared to have an adverse effect and induced a leftward shift in the survival curve.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. A method for the treatment of amyotrophic lateral sclerosis (ALS), comprising administering to a patient in need of such treatment a compound of general formula (I): 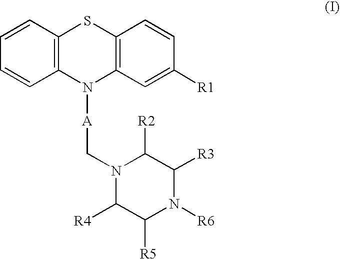
wherein
A represents a straight or branched alkylene chain of from 2 to 6 carbon atoms separating the nitrogen atoms linked thereto by at least two carbon atoms;
R1 represents hydrogen, halogen, lower alkyl,lower alkoxyl, lower alkanoyl, lower alkyl-mercapto, trifluoromethylmercapto, lower alkyl-sulfonyl (preferably methylsulfonyl), perfluoroalkyl of 1 to 3 carbon atoms;
R2, R3, R4 and R5 each represent methyl, ethyl or hydrogen,
R6 represents hydrogen, lower alkyl, lower alkyl substituted by halogen, hydroxy-lower-alkyl or aliphatic acyloxy-lower-alkyl having 1 to 4 carbon atoms in the acyloxy portion and 1 to 6 carbon atoms in the alkyl portion, CH₂—CH₂—OR7 where R7 represents hydrogen, COR8 where R8 is a branched or straight chain alkyl radical of from seven to fourteen carbon atoms.

14. A method according to claim 13 wherein
A represents ethylene, propylene or 2-methylpropylene;
R1 represents hydrogen, chloro, COCH₃, —CF₃;
R2, R3, R4 and R5 each represent hydrogen;
R6 represents CH₃, CH₂—CH₂—O—R7 where R7 represents hydrogen, COR8 where R8 is a straight chain alkyl radical of from seven to fourteen carbon atoms.

15. A method according to claim 13 wherein the compound of general formula (I) is flufenazine, or a pharmaceutically acceptable salt or ester thereof.

16. A method according to claim 13, wherein the medicament is for oral, rectal, subcutaneous, intramuscular or intravascular administration route.

17. A method according to claim 13, wherein the medicament comprises as active ingredient from 0.2 mg to 500 mg of the compound of general formula (I).

18. A method according to claim 13, wherein the medicament is administered at doses comprised between 0.1 mg/kg to 10 mg/kg.
